(19) United States
(12) Patent Application Publication
(10) Pub. No.: US 2007/0117729 A1
(43) Pub. Date: May 24, 2007

(54) Multi-phase personal care composition comprising a depositing perfume

(76) Inventors: Rebecca Ann Taylor, Cincinnati, OH (US); Jianjun Justin Li, West Chester, OH (US); Virginia Tzung-Hwei Hutchins, Cincinnati, OH (US); Edward Dewey Smith III, Mason, OH (US); Zerlina Gudzar Dubois, Mason, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/590,977

(22) Filed: Nov. 1, 2006

Related U.S. Application Data
(60) Provisional application No. 60/732,092, filed on Nov. 1, 2005.

(51) Int. Cl. C11D 3/50; A61K 8/00

(52) U.S. Cl. 510/130; 510/101

(57) ABSTRACT

A personal care composition and multi-phase personal care composition are described that comprise 3% or less, by weight of the personal care composition, of a perfume. The personal care composition comprises at least 0.27%, by weight of the personal care composition, of the perfume raw materials having a Kovat's Index of greater than 1700.

### CROSS REFERENCE TO RELATED APPLICATION

This application claims the benefit of U.S. Provisional Application No. 60/732,092, filed Nov. 1, 2005.

### FIELD OF THE INVENTION

The present invention relates to a personal care composition and a multiphase personal care composition that comprises 3% or less, of a perfume, and at least 0.27%, by weight of the personal care composition, of the perfume raw materials having a Kovat's Index of greater than 1700.

### BACKGROUND OF THE INVENTION

Personal care compositions are well known and widely used. In order to be acceptable to consumers, a personal care composition must exhibit good cleaning properties, must exhibit good lathering characteristics, must be mild to the skin (not cause drying or irritation) and preferably should even provide a conditioning benefit to the skin. Moreover, odor or scent is a characteristic of personal care compositions which drives consumer acceptance. Some consumers choose a personal care composition for both the odor of the product itself, as well as, the residual odor the composition leaves on the skin or hair. Perfumes contain individual components called perfume raw materials (PRMs), which add to the perfume's scent. Certain PRMs are more volatile and provide a scent or “bloom” when the bottle is opened, or when used in the shower. Other PRMs tend to deposit on the skin and can last longer, providing a residual benefit, or perfume longevity. These PRMs are sometimes called ‘base notes’ of a fragrance.

The residual odor is the scent of the product on the consumer's skin. Some consumers prefer a personal care composition that comprises perfumes with longevity, so that the scent lasts on the skin leaving a residual scent benefit. Personal care compositions are known and widely used that have a product scent and leave a residual odor of the composition on the skin or hair. It is also known that enhanced longevity can be obtained with conventional fragrances merely by increasing the level of perfume in a composition, such as by increasing the level of perfume to 4% or even higher. The increased level of perfume, however, can affect other qualities of the composition, such as viscosity, lather and visual appearance. For example, the perfume can interact with surfactant micelles, increasing viscosity of the personal care composition and reducing the lather obtained from the personal care composition. As well, compositions that comprise surfactant also have limited ability to absorb perfume, and high levels of perfume can form a separate, turbid phase in an otherwise transparent composition making the composition visually unattractive to some consumers. Moreover, increasing the perfume level increases the cost of the personal care composition. So, achieving increased longevity with less perfume is generally desirable.

Accordingly, the need still remains for a multi-phase personal care composition that provides cleansing with increased perfume longevity without high levels of perfumes present in the composition.

### SUMMARY OF THE INVENTION

The present invention relates to a personal care composition comprising 3% or less by weight of said personal care composition, of a perfume. The personal care composition comprises at least 0.27%, by weight of personal care composition, of perfume raw materials having a Kovat's Index of greater than 1700.

The present invention also relates to a multi-phase, personal care composition comprising a first phase and a second phase. The multi-phase personal care composition comprises a perfume. The personal care composition comprises at least 0.27%, by weight of the personal care composition, of perfume raw materials that have a Kovat's Index of greater than 1700.

The Inventors of the present invention believe that enhanced perfume longevity can be obtained by preparing a perfume ‘accord’ that comprises a high proportion of individual PRMs that are individually inclined to longevity. The longevity benefit can be obtained at a lower cost and a desirable viscosity and latherability to consumers due to the lower total perfume level.

### DETAILED DESCRIPTION OF THE INVENTION

The term “ambient conditions” as used herein, refers to surrounding conditions at one (1) atmosphere of pressure, 50% relative humidity, and 25° C.

“Kovat's Index” (KI, or Retention Index) is defined by the selective retention of solutes or perfume raw materials (PRMs) onto a chromatograph column. It is primarily determined by the column stationary phase and the properties of solutes or PRMs. For a given column system, a PRMs polarity, molecular weight, vapor pressure, boiling point and the stationary phase property determine the extent of retention. To systematically express the retention of analyte on a given GC column, a measure called Kovat's Index (or retention index) is defined. Kovat's Index (KI) places the volatility attributes of an analyte (or PRM) on a column in relation to the volatility characteristics of n-alkane series on that column. Typical columns used are DB-5 and DB-1.

By this definition the KI of a normal alkane is set to 100 n, where n=number of carbons atoms of the n-alkane. With this definition, the Kovat's index of a PRM, x, eluting at time t′, between two neighboring n-alkanes with number of carbon atoms n and N having corrected retention times t′ₙ and t′_{N} respectively will then be calculated as:

$$KI = 100 ( n + \frac{log {t}_{x}^{′} - log {t}_{n}^{′}}{log {t}_{N}^{′} - log {t}_{n}^{′}} ) \qquad ( 1 )$$

By the term “multi-phase” as used herein, is meant that the phases of the present compositions occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal care compositions can comprise at least two visually distinct phases which are present within the container as a visually distinct pattern. The pattern results from the combination of the “multi-phase” composition by a process herein described. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof. The phases may be various different colors, and/or include particles, glitter or pearlescent agents in at least one of the phases in order to offset its appearance from the other phase(s) present.

The term “personal care composition” as used herein, refers to compositions intended for topical application to the skin or hair. Non-limiting examples of personal care compositions include compositions selected from the group consisting of bar soap, body wash, shampoo, conditioner, in-shower body moisturizer and mixtures thereof.

The term “structured,” as used herein means having a rheology that confers stability on the multi-phase composition. The degree of structure is determined by the Yield Stress and Zero Shear Viscosity Method and by the Ultracentrifugation Method, both described hereafter. When a phase is a structured phase, typically it has a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter. When a phase is a structured phase, it may also typically have a Zero Shear Viscosity of at least about 500 Pascal-seconds (Pa-s), preferably at least about 1,000 Pa-s, more preferably at least about 1,500 Pa-s, even more preferably at least about 2,000 Pa-s. Accordingly, when a cleansing phase or a surfactant phase of the multi-phase composition of the present invention is structured, it has a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

The term “visually distinct phase” as used herein, refers to a region of the multi-phase personal care composition having one average composition, as distinct from another region having a different average composition, wherein the regions are visible to the unaided naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase may also be constituted or re-constituted, collected, or separated into a bulk phase in order to observe its properties, e.g., by centrifugation, filtration or the like.

The present invention relates to a personal care composition comprising 3% or less by weight of said personal care composition, of a perfume. The personal care composition comprises at least 0.27%, by weight of the personal care composition, of perfume raw materials having a Kovat's Index of greater than 1700.

The total perfume concentration in the personal care composition can range from about 0.001% to 3%. The personal care composition and the multi-phase personal composition comprise 3% or less, by weight of said personal care composition, of a perfume. The personal care composition and multi-phase personal care composition can comprise 2.5% or less, 2% or less, 1.5% or less, 1% or less, or 0.5% or less, by weight of the personal care composition, of a perfume.

Examples of perfumes of the present invention are illustrated in Table 1.

TABLE 1
Perfumes of the Prior Art and the Present Invention

| Perfume | | | Prior | | | | |
|---|---|---|---|---|---|---|---|
| Raw | | Kovat's | Art Perfume | | | | |
| Material | INCI Name | Index | 1% wt. | 2% wt. | 3% wt. | 4% wt. | 5% wt. |
| Hexyl | 2-hydroxy- | 1709 | 4 | — | — | 5 | 10 |
| Salicylate | benzoic acid, | | | | | | |
| | hexyl ester | | | | | | |
| LRG 201 | Methyl 2,4- | 1735 | 0.1 | — | 0.1 | — | — |
| | dihydroxy-3,6- | | | | | | |
| | dimethylbenzoate | | | | | | |
| Hexyl | Octanal, 2- | 1772 | 8 | 19.05 | 10 | 20 | 25 |
| Cinnamic | (phenylmethylene) | | | | | | |
| Aldehyde | | | | | | | |
| Galaxolide | Hexamethylindano | 1910 | 10 | 23.82 | — | — | 15 |
| | pyran | | | | | | |
| Delta | 3-Methylcyclo- | 1917 | 0.1 | — | — | — | — |
| Muscenone | pentadecenone | | | | | | |
| Ambrettolide | Oxacycloheptandec- | 2005 | 1 | — | 5 | 5 | 5 |
| | 10-en-2- | | | | | | |
| | one | | | | | | |
| Ambrofix | Dodecahydro- | 1772 | 0.5 | — | — | 0.5 | — |
| | 3a,6,6,9a- | | | | | | |
| | tetramethyl- | | | | | | |
| | naphtho(2,1-b)- | | | | | | |
| | furan | | | | | | |
| Indolene | Indol/Hydroxy- | less | 0.01 | — | — | — | — |
| | citronellal Schiff | than | | | | | |
| | base | >2000 | | | | | |
| Exaltolide | Cyclopenta- | 1891 | — | — | 10 | 10 | — |
| | decanolide | | | | | | |
| Benzyl | Benzoic acid, 2- | 1905 | — | — | 5 | 5 | 5 |
| Salicylate | hydroxy-, | | | | | | |
| | phenylmethyl | | | | | | |
| | ester | | | | | | |
| Other | | greater | 76.29 | 57.13 | 69.9 | 54.5 | 40 |
| Perfume | | than | | | | | |
| Raw | | <1700 | | | | | |
| Materials | | | | | | | |

The percentage of perfume raw materials having a Kovat's Index of greater than 1700 of the composition of the present invention can range from 0.27% to about 0.8%, by weight of the personal care composition. The personal composition and multi-phase personal care composition can comprise at least 0.27%, at least 0.3%, at least 0.35%, at least 0.4%, at least 0.5%, at least 0.6% or at least 0.7%, of perfume raw materials having a Kovat's Index of greater than 1700 in some embodiments of the present invention.

Examples of perfume raw materials useful for the present invention are illustrated in Table 2.

TABLE 2
Perfume Raw Materials Useful for the Present Invention

| Perfume Raw | | Kovat's | Boiling | |
|---|---|---|---|---|
| Material | INCI Name | Index | Point | ClogP |
| Hexyl | 2-hydroxy-benzoic acid, hexyl | 1709 | 304.3 ± 15.0 | 4.891 ± 0.241 |
| Salicylate | ester | | | |
| LRG 201 | Methyl 2,4-dihydroxy-3,6- | 1735 | 319.8 ± 7.0 | 2.839 ± 0.257 |
| | dimethylbenzoate | | | |
| Hexyl Cinnamic | Octanal, 2-(phenylmethylene)- | 1772 | 308.1 ± 0.0 | 5.332 ± 0.374 |
| Aldehyde | | | | |
| Galaxolide | Hexamethylindano pyran | 1910 | 326.3 ± 11.0 | 5.947 ± 0.311 |
| Delta | 3-Methylcyclopentadecenone | 1917 | 329.5 ± 10.0 | 6.333 ± 0.255 |
| Muscenone | | | | |
| Ambrettolide | Oxacycloheptandec-10-en-2-one | 2005 | 399.2 ± 27.0 | 5.516 ± 0.287 |
| Ambrofix | Dodecahydro-3a,6,6,9a- | 1797 | 273.9 ± 8.0 | 5.411 ± 0.295 |
| | tetramethylnaphtho(2,1-b)-furan | | | |
| Indolene | Indol/Hydroxycitronellal Schiff | >2000 | 488.1 | 6.745 |
| | base | | | |
| Exaltolide | Cyclopentadecanolide | 1891 | 344.8 ± 10.0 | 5.443 ± 0.277 |
| Benzyl | Benzoic acid, 2-hydroxy-, | 1905 | 320.0 ± 0.0 | 4.007 ± 0.254 |
| Salicylate | phenylmethyl ester | | | |
| Ethylene | 1,4-dioxacycloheptadecane-5,17- | 2060 | 476.1 ± 28.0 | 2.896 ± 0.399 |
| Brassylate | dione | | | |
| Nectaryl | 2-[2-(4-methyl-3-cyclohexen-1- | 1826 | 318.2 ± 27.0 | 4.259 ± 0.266 |
| | yl)propyl]-cyclopentanone | | | |
| Okoumal | 1,3-dioxolane,2,4-diemthyl-2- | 1850 | 352.8 ± 27.0 | 5.744 ± 0.429 |
| | (5,6,7,8-tetrahydro-5,5,8,8- | | | |
| | tetramethyl-2-naphthaleneyl)- | | | |

A perfume raw material (PRM) is characterized by its lipophilicity as determined by its calculated log₁₀(octanol/water partition coefficient), or ClogP. The ClogP of a PRM is the calculated ratio between its equilibrium concentrations in octanol and in water in a mixture comprising only octanol, water and the PRM. The preferred PRMs of this invention have a calculated octanol/water partition coefficient P of about 100 or higher. Since the calculated partition coefficients of the preferred PRMs of this invention have high values, they are more conveniently given in the form of their logarithm to the base 10, ClogP. Thus the preferred PRMs of this invention have ClogP at 25° C. of greater than about 3.5.

ClogP is a calculated quantity for a PRM, determined from a mathematical algorithm using molecular substructure or fragment contributions with correction factors. The approach is common in such fields as toxicology, environmental transport, and pharmaceuticals, for example to facilitate development of drugs, especially for topical drugs that interact with lipid bilayers in skin, a molecular mechanism not dissimilar to interaction of PRM molecules with surfactant. Different substructure fragment algorithms exist which can calculate different ClogP values for the same molecule, based on differences in algorithms and/or coefficients, as can be found in scientific literature. For the purposes of our invention, ClogP of a PRM is determined using the algorithm from Advanced Chemistry Development Labs as referenced and updated in the scientific literature (Hansch, C. and Leo, A., Substituent Constants for Correlation Analysis in Chemistry and Biology, Wiley Interscience New York (1979); updated in Leo., A. and Hoekman, D., Perspect. in Drug Discov. & Design, 18, 19 (2000)), using the software Solaris V4.67. The software is published by Daylight Chemical Information Systems, Santa Fe, N. Mex., USA (U.S. phone 505-989-1000) and is downloadable from the web site http://www.daylight.com/download/.

Most common perfume ingredients which are derived from natural sources are composed of a multitude of components. For example, orange terpenes contain about 90% to about 95% d-limonene, but also contain many other minor ingredients. When each such material is used in the formulation of perfumes of the present invention, it is counted as one ingredient (i.e., one PRM), for the purpose of defining the invention. Synthetic reproductions of such natural perfume ingredients are also comprised of a multitude of components, e.g. isomers, and are counted as one ingredient for the purpose of defining the invention. The Kovat's Index, ClogP, and other properties are the properties of the major chemical component.

In the perfume art, some auxiliary materials having no odor, or a low odor, are used, e.g., as solvents, diluents, extenders or fixatives. Non-limiting examples of these materials are ethyl alcohol, carbitol, glycols such as dipropylene glycol, diethyl phthalate, triethyl citrate, isopropyl myristate, and benzyl benzoate. These materials are used for, e.g., solubilizing or diluting some solid or viscous perfume ingredients to, e.g., improve handling and/or formulating. These materials are useful in the perfumes, but are not counted in the calculation of the limits for the definition/formulation of the perfumes of the present invention.

The Inventors believe that enhanced perfume longevity can be obtained by preparing a perfume ‘accord’ that comprises a high proportion of individual PRMs that are individually inclined to longevity. The longevity benefit can be obtained at a lower cost with a desirable viscosity and ability to lather to consumers due to the lower total perfume level. Specifically, the inventors believe they have demonstrated increased perfume deposition from an accord having a high proportion of components with Kovats Index greater than 1700.

The body wash compositions in Table 3 below contain water, sodium trideceth sulfate, sodium lauryl sulfate, sodium chloride, sodium lauroamphoacetate, iso-steareth-2, xanthum gum, acrylates vinyl isodecanoate crosspolymer, triethanolamine, citric acid, DMDM hydantoin, guar hydroxypropyltrimonium chloride, disodium EDTA, sodium benzoate, PEG 90M, methyl chloro isothiazolinone & methyl isothiazolinone, D&C green 3, FD&C Blue 1. The body washes also comprise the perfume compositions illustrated in Table 1.

TABLE 3
Comparison of the Present Invention with Prior Art Compositions

| | | | | Amount |
|---|---|---|---|---|
| | | % of | % of | of |
| | Compositions including | Perfume | Composition | PRMs |
| | amount of Perfume | with | with | deposited |
| Compositions | within the composition | KI >1700 | KI >1700 | on skin |
| Present | Body wash w/1.245% | 42.87% | 0.534% | 0.622 ug/cm² |
| invention | Perfume 2 | | | |
| Prior Art | Body wash w/1% | 23.71% | 0.237% | 0.301 ug/cm² |
| composition 1 | Perfume 1 | | | |
| Prior Art | Body wash w/4% | 23.71% | 0.948% | 1.186 ug/cm² |
| composition 2 | Perfume 1 | | | |

Table 3 compares the compositions of the present invention to those in the prior art. The compositions of the present invention comprise 3% or less, by weight of the personal care composition, of a perfume, and at least 0.27%, by weight of the personal care composition, of the perfume raw materials having a Kovat's Index of greater than 1700. The prior art composition 1 comprises a perfume at 1%, by weight of the personal care composition. The deposition data show that the composition of the present invention deposits a higher concentration of PRMs on the skin, than the prior art composition 1 with a comparable concentrations of perfume. The prior art composition 2 comprises a perfume at 4%, by weight of the personal care composition and shows the know trend that the concentration of the perfume increases deposition on the skin.

The present invention also relates to a multi-phase, personal care composition comprising a first phase and a second phase. The multi-phase personal care composition comprises a perfume. The perfume comprises perfume raw materials wherein at least 0.27% of said perfume raw materials having a Kovat's Index of greater than 1700.

The multi-phase personal care composition of the present invention is typically extrudable or dispensible from a package. The multi-phase personal care compositions typically exhibit a viscosity of from about 1,500 centipoise (cP) to about 1,000,000 cP, as measured by the Viscosity Method as described in copending application Ser. No. 10/841,174 filed on May 7, 2004 titled “Multi-phase Personal Care Compositions.”

When evaluating a multi-phase personal care composition, by the methods described herein, preferably each individual phase is evaluated prior to combining, unless otherwise indicated in the individual methodology. However, if the phases are combined, each phase can be separated by centrifugation, ultracentrifugation, pupating, filtering, washing, dilution, concentration, or combination thereof, and then the separate components or phases can be evaluated. Preferably, the separation means is chosen so that the resulting separated components being evaluated is not destroyed, but is representative of the component as it exists in the multi-phase personal care composition, i.e., its composition and distribution of components therein is not substantially altered by the separation means. Generally, multi-phase compositions comprise domains significantly larger than colloidal dimensions so that separation of the phases into the bulk is relatively easy to accomplish while retaining the colloidal or microscopic distribution of components therein. Preferably, the compositions of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

The multi-phase personal care compositions of the present invention can comprise at least two visually distinct phases, wherein the composition can have a first structured phase, a second phase, a third phase, a fourth phase and so on. The ratio of a first phase to a second phase is preferably from about 1:99 to about 99:1, preferably from about 90:10 to about 10:90, more preferably from about 80:20 to about 20:80, even more preferably from about 70:30 to about 30:70, still even more preferably from about 60:40 to about 40:60, even still even more preferably about 50:50. The preferred pH range of the multi-phase personal care composition is from about 5 to about 8. Each phase could be one or more of the following no limiting examples including: a cleansing phase, a benefit phase, and a non-lathering structured aqueous phase, which are described in greater detail hereinafter.

In embodiments where the personal care composition is multi-phase, the first phase or second phase of the multi-phase personal care composition of the present invention can be a cleansing phase. Preferably, the surfactant component comprises a mixture of surfactants. The multi-phase personal care composition typically comprises from about 1% to about 99%, by weight of the composition, of said cleansing phase.

The surfactant component preferably comprises a lathering surfactant or a mixture of lathering surfactants. The surfactant component comprises surfactants suitable for application to the skin or hair. Suitable surfactants for use herein include any known or otherwise effective cleansing surfactant suitable for application to the skin, and which are otherwise compatible with the other essential ingredients in the multi-phase personal care composition including water. These surfactants include anionic, nonionic, cationic, zwitterionic, amphoteric surfactants, soap, or combinations thereof. Preferably, anionic surfactant comprises at least 40% of the surfactant component, more preferably from about 45% to about 95% of the surfactant component, even more preferably from about 50% to about 90%, still more preferably from about 55% to about 85%, and even still most preferably at least about 60% of the surfactant component comprises anionic surfactant.

The personal care compositions of present invention preferably comprises a surfactant component at concentrations ranging from about 2% to about 90%, more preferably from about 3% to about 30%, even more preferably from about 4% to about 25%, still more preferably from about 5% to about 20%, still even more preferably from about 10% to about 20%, and even still even more preferably from about 15% to about 20%, by weight of the first phase.

The surfactant component is preferably a structured domain comprising surfactants. The structured domain enables the incorporation of high levels of benefit components in a separate phase that are not emulsified in the composition. In a preferred embodiment the structured domain is an opaque structured domain. The opaque structured domain is preferably a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al. on Dec. 30, 1975.

Preferred linear anionic surfactants for use in the surfactant component of the multi-phase, personal care composition include ammonium lauryl sulfate, ammonium laureth sulfate, sodium lauryl sulfate, sodium laureth sulfate, sodium lauroyl sacrosanct, and combinations thereof.

Branched anionic surfactants and monomethyl branched anionic surfactants suitable for the present invention are described in commonly owned U.S. Application Ser. No. 60/680,149 entitled “Structured Multi-phased Personal Cleansing Compositions Comprising Branched Anionic Surfactants” filed on May 12, 2004 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ parity sulfate and sodium C₁₂₋₁₃ parity-n sulfate. Branched surfactants can be derived from synthetic alcohols such as the primary alcohols from the liquid hydrocarbons produced by Fischer-Tropic condensed singes, for example Safol™ 23 Alcohol available from Sasol North America, Houston, Tex.; from synthetic alcohols such as Neodol™ 23 Alcohol available from Shell Chemicals, USA; from synthetically made alcohols such as those described in U.S. Pat. No. 6,335,312 issued to Coffindaffer, et al. on Jan. 1, 2002. Sulfates can be prepared by conventional processes to high purity from a sulfur based SO₃ air stream process, chlorosulfonic acid process, sulfuric acid process, or Oleum process.

Amphoteric surfactants suitable for use in the personal care compositions of the present invention, include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, and N-alkyltaurines such as the one prepared by reacting dodecylamine with sodium isethionate according to the teaching of U.S. Pat. No. 2,658,072 issued to Kosmin, et al. Amphoacetates and diamphoacetates, may also be used. Sodium lauroamphoacetate, sodium cocoamphoacetetate, disodium lauroamphoacetate, and disodium cocodiamphoacetate are preferred in some embodiments.

Zwitterionic surfactants suitable for use in the personal care compositions of the present invention include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Other zwitterionic surfactants suitable for use in the multi-phase, personal care composition include betaines, such as, cocoamidopropyl betaine, cocobetaine. and carboxymethyl betaine.

Non-limiting examples of preferred nonionic surfactants for use herein are those selected form the group consisting of C₈-C₁₄ glucose amides, C₈-C₁₄ alkyl polyglucosides, sucrose laurate, alkanolamides, ethoxylated alcohols and mixtures thereof. In a preferred embodiment the nonionic surfactant is selected from the group consisting of glyceryl monohydroxystearate, steareth-2, isosteareth-2, hydroxy stearic acid, propylene glycol stearate, PEG-2 stearate, sorbitan monostearate, glyceryl stearate, laureth-2, cocamide monoethanolamine, lauramide monoethanolamine, and mixtures thereof.

Mixtures of anionic surfactants may be used in some embodiments, including mixtures of linear and branched surfactants, and anionic surfactants with nonionic, amphoteric, and/or zwitterionic surfactants.

The electrolyte, if used, can be added per se to the personal care composition of the present invention or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the surfactant component of the composition in the amount of from about 0.1% to about 15% by weight, preferably from about 1% to about 6% by weight of the multi-phase personal care composition, but may be varied if required.

In one embodiment of the personal care composition of the present invention comprises a surfactant component comprising a mixture of at least one nonionic surfactant, at least one anionic surfactant and at least one amphoteric surfactant, and an electrolyte. In another one embodiment, the surfactant can comprise a mixture of surfactants, water, at least one anionic surfactant, an electrolyte, and at least one alkanolamide. The amount of alkanolamide in the composition is typically from about 0.1% to about 10%, preferably from about 2% to about 5%, by weight of the cleansing phase.

The first phase or second phase of the multi-phase, personal care compositions of the present invention can be a benefit phase. The benefit phase in the present invention is preferably anhydrous. The benefit phase typically comprises hydrophobic materials. The benefit phase comprises from about 1% to about 100%, preferably at least about 35%, most preferably at least about 50%, by weight of the benefit phase, of a hydrophobic material. The hydrophobic materials suitable for use in the present invention preferably have a Vaughan Solubility Parameter of from about 5 to about 15 (cal/cm³)^{1/2}, as defined by Vaughan in Cosmetics and Toiletries, Vol. 103. Non-limiting examples of hydrophobic materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic compositions are preferably selected among those having defined rheological properties as described hereinafter, including selected Consistency value (K) and Shear Index (n). These preferred rheological properties are especially useful in providing the multi-phase, personal care compositions with improved deposition of hydrophobic materials. The benefit phase has a Consistency Value (K) from about 20 to about 2,000 Pa-s, preferably from about 25 to about 500 Pa-s, more preferably from about 30 to about 450 Pa-s, still more preferably from about 30 to about 400 Pa-s and even still more preferably from about 30 to about 350 Pa-s. The benefit phase has a Shear Index from about 0.025 to about 0.99, preferably from about 0.05 to about 0.70 and more preferably from about 0.09 to about 0.60.

Nonlimiting examples of hydrophobic material suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, and combinations thereof.

The benefit phase of the composition preferably can comprise one or more hydrophobic materials, wherein at least 1% by weight of the hydrophobic materials is selected the group consisting of petrolatum, mineral oil, triglycerides such as sunflower seed oil, alkyl siloxanes, polymethylsiloxanes and methylphenylpolysiloxanes, and mixtures thereof. More preferably, at least about 20% by weight of the hydrophobic materials are selected from the groups of petrolatum, mineral oil, paraffins, polyethylene, polydecene, dimethicones, alkyl siloxanes, lanolins, triglycerides. More preferably, at least about 50% by weight of the hydrophobic materials are selected from the groups of petrolatum, mineral oil, paraffins, polyethylene, polydecene, dimethicones, alkyl siloxanes, lanolins, and triglycerides.

Examples of suitable benefit phases and description of measuring the values of Consistency (K) and Shear Index (n) are described in U.S. patent application Ser. No. 10/665,670, Publication No. 2004/0057920 A1 entitled Striped liquid personal cleansing compositions containing a cleansing phase and a separate benefit phase” filed by Fact, et al. on Sep. 18, 2003, published on Apr. 4, 2004, U.S. patent application Ser. No. 10/699,469 Publication No. 2004/0092415 A1 entitled “Striped liquid personal cleansing compositions containing a cleansing phase and a separate benefit phase with improved stability” filed by Fact, et al. on Oct. 31, 2003, published on May 13, 2004 and U.S. patent application Ser. No. 10/837,214 Publication No. 2004/0219119 A1 entitled “Visually distinctive multiple liquid phase compositions” filed by Weir, et al. on Apr. 30, 2004, published on Nov. 18, 2004.

The first phase or second phase of the multi-phase personal care compositions of the present invention can comprise a structured aqueous phase that comprises a water structurant and water. The structured aqueous phase can be hydrophilic and in a preferred embodiment the structured aqueous phase is a hydrophilic, non-lathering gelled water phase. In addition, the structured aqueous phase typically comprises less than about 5%, preferably less than about 3%, and more preferably less than about 1%, by weight of the structured aqueous phase, of a surfactant. In one embodiment of the present invention, the structured aqueous phase is free of lathering surfactant in the formulation.

The structured aqueous phase of the present invention can comprise from about 30% to about 99%, by weight of the structured aqueous phase, of water. The structured aqueous phase generally comprises more than about 50%, preferably more than about 60%, even more preferably more than about 70%, still more preferably more than about 80%, by weight of the structured aqueous phase, of water.

The structured aqueous phase will typically have a pH of from about 5 to about 9.5, more preferably about 7. A water structurant for the structured aqueous phase can have a net cationic charge, net anionic charge, or neutral charge. The structured aqueous phase of the present compositions can further comprise optional ingredients such as, pigments, pH regulators (e.g. triethanolamine), and preservatives.

The structured aqueous phase can comprise from about 0.1% to about 30%, preferably from about 0.5% to about 20%, more preferably from about 0.5% to about 10%, and even more preferably from about 0.5% to about 5%, by weight of the structured aqueous phase, of a water structurant.

The water structurant is typically selected from the group consisting of inorganic water structurants, charged polymeric water structurants, water soluble polymeric structurants, associative water structurants, and mixtures thereof. Non-limiting examples of inorganic water structurants include silicas, polymeric gellants such as polyacrylates, polyacrylamides, starches, modified starches, crosslinked polymeric gellants, copolymers, and mixtures thereof. Non-limiting examples of charged polymeric water structurants for use in the multi-phase personal care composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), and mixtures thereof. Non-limiting examples of water soluble polymeric structurants for use in the multi-phase personal care composition include cellulose gums and gel, and starches. Non-limiting examples of associative water structurants for use in the multi-phase personal care composition include xanthum gum, gellum gum, pectins, alginates such as propylene glycol alginate, and mixtures thereof.

The phases of the multi-phase personal care composition, preferably the cleansing phase, can further comprise a polymeric phase structurant. The compositions of the present invention typically can comprise from about 0.05% to about 10%, preferably from about 0.1% to about 4% and more preferably from about 0.2% to about 2% by weight of the phase, of a polymeric phase structurant. Non-limiting examples of polymeric phase structurant include but is not limited to the following examples: deflocculating polymers, naturally derived polymers, synthetic polymers, crosslinked polymers, block polymers, block copolymers, copolymers, hydrophilic polymers, nonionic polymers, anionic polymers, hydrophobic polymers, hydrophobically modified polymers, associative polymers, oligomers, and copolymers thereof as described in U.S. Patent Application No. 60/628,036 filed on Nov. 15, 2003 by Wagner, et al. titled “Depositable Solids.” Preferably the polymeric phase structurant can be crosslinked. These polymeric phase structurant useful in the present invention are more fully described in U.S. Pat. No. 5,087,445, to Haffey et al., issued Feb. 11, 1992; U.S. Pat. No. 4,509,949, to Huang et al., issued Apr. 5, 1985, U.S. Pat. No. 2,798,053, to Brown, issued Jul. 2, 1957. See also, CTFA International Cosmetic Ingredient Dictionary, fourth edition, 1991, pp. 12 and 80.

The phases of the present compositions, preferably the cleansing phase, optionally can further comprise a liquid crystalline phase inducing structurant, which when present is at concentrations ranging from about 0.3% to about 15%, by weight of the phase, more preferably at from about 0.5% to about 5% by weight of the phase. Suitable liquid crystalline phase inducing structurants include fatty acids (e.g. lauric acid, oleic acid, isostearic acid, linoleic acid) ester derivatives of fatty acids (e.g. propylene glycol isostearate, propylene glycol oleate, glyceryl isostearate) fatty alcohols, trihydroxystearin (available from Rheox, Inc. under the trade name THIXCIN® R). Preferably, the liquid crystalline phase inducing structurant is selected from lauric acid, trihydroxystearin, lauryl pyrrolidone, and tridecanol.

The multi-phase personal care compositions of the present invention can additionally comprise an organic cationic deposition polymer in the one or more phases as a deposition aid for the benefit agents described herein. Suitable cationic deposition polymers for use in the compositions of the present invention contain cationic nitrogen-containing moieties such as quaternary ammonium or cationic protonated amino moieties. The cationic protonated amines can be primary, secondary, or tertiary amines depending upon the particular species and the selected pH of the multi-phase personal care composition. Suitable cationic deposition polymers that would be useful in the compositions of the present invention are disclosed in the co-pending and commonly assigned U.S. Patent Application No. 60/628,036 filed on Nov. 15, 2003 by Wagner, et al. titled “Depositable Solids.”

Nonlimiting examples of cationic deposition polymers for use in compositions include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M.

The personal care compositions of the present invention can comprise a particle. A water insoluble particle of various shapes and densities can be useful. In a preferred embodiment, the particle tends to have a spherical, an oval, an irregular, or any other shape in which the ratio of the largest dimension to the smallest dimension (defined as the Aspect Ratio) is less than about 10, preferably less than about 8, and still more preferably the Aspect Ratio of the particle is less than about 5. Preferably, the particle will also have physical properties which are not significantly affected by typical processing of the composition.

The personal care compositions of the present invention can comprise an exfoliant particle selected from the group consisting of polyethylene, microcrystalline wax, jojoba esters, amorphous silica, talc, tricalcium orthophosphate, and mixtures thereof. Exfoliant particles can be comprised in at least one phase of the multi-phase personal care composition at a level of less than about 10%, by weight of the composition.

The personal care compositions of the present invention can comprise a shiny particle in at least one phase of the multi-phase personal care composition. Nonlimiting examples of shiny particles include the following: interference pigment, multi-layered pigment, metallic particle, solid and liquid crystals, and combinations thereof. An interference pigment is a pigment with pearl gloss prepared by coating the surface of a particle substrate material with a thin film. Interference pigments and hydrophobically modified interference pigments are suitable for use in the compositions of the present invention.

The personal care compositions of the present invention can comprise beads of any color and may be located in one or more phases of the of the multi-phase personal care composition. Suitable beads include those known in the art, including soft and hard beads. Suitable examples of soft beads include unispheres, made by Induchem, Unispheres NT-2806 (Pink). Suitable examples of hard beads include polyethylene or oxidized polyethylene, preferably those made by Accutech.

One or more of the phases of the multi-phase personal care composition can comprise a variety of additional optional ingredients. Such optional ingredients are most typically those materials approved for use in cosmetics and that are described in reference books such as the CTFA Cosmetic Ingredient Handbook, Second Edition. The Cosmetic, Toiletries, and Fragrance Association, Inc. 1988, 1992.

Other non limiting examples of these optional ingredients include vitamins and derivatives thereof (e.g., ascorbic acid, vitamin E, tocopheryl acetate, and the like), sunscreens; thickening agents (e.g., polyol alkoxy ester, available as Crothix from Croda), preservatives for maintaining the anti microbial integrity of the cleansing compositions, anti-acne medicaments (resorcinol, salicylic acid, and the like), antioxidants, skin soothing and healing agents such as aloe vera extract, allantoin and the like, chelators and sequestrants, skin lightening agents, and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

### Test Methods

Yield Stress and Zero Shear Viscosity Method: The Yield Stress and Zero Shear Viscosity of a phase of the present composition, can be measured either prior to combining in the composition, or after combining in the composition by separating the phase by suitable physical separation means, such as centrifugation, pipetting, cutting away mechanically, rinsing, filtering, or other separation means.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress.

First a sample of the phase is obtained and placed in position on the rheometer base plate, the measurement geometry (upper plate) moving into position 1 mm above the base plate. Excess phase at the geometry edge is removed by scraping after locking the geometry. If the phase comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a phase has any particle larger than 5 mm in any dimension, the particles are removed prior to the measurement.

The determination is performed via the programmed application of a continuous shear stress ramp from 0.1 Pa to 1,000 Pa over a time interval of 5 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:
Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).
%variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

Ultracentrifugation Method: The Ultracentrifugation Method is used to determine the percent of a structured domain or an opaque structured domain that is present in a multi-phase personal care composition that comprises a cleansing phase comprising a surfactant component. The method involves the separation of the composition by ultracentrifugation into separate but distinguishable layers. The multi-phase personal care composition of the present invention can have multiple distinguishable layers, for example a non-structured surfactant layer, a structured surfactant layer, and a benefit layer.

First, dispense about 4 grams of multi-phase personal care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:
Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacing of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=H_{c}/Hₛ*100%

If there is no benefit phase present, use the total height as the surfactant layer height, Hₛ=Hₐ.

Perfume Deposition Analysis Using a Large Volume Injection System-Gas Chromatography/Mass Spectrometry: The Perfume Deposition Analysis is used to determine the amount of residual perfume components on the skin after application of a personal care composition that comprises a perfume. The amount of the perfume component is analyzed using a cooled-injection system for large volume injection Gas Chromatograph-Mass Spectrometer (GC-MS.) All laboratory instruments used in this analysis should be operated according to manufacturers' instructions, as set forth in the instrument operation manuals and instructional materials, unless otherwise indicated.

Prior to determination of the amount of residual perfume in the personal care composition, a human subject must have the personal care composition applied and washed off the skin. Before washing the forearm of a human subject, use a plastic template (area 119 cm²) to mark the area that will be treated with a personal care composition. Next, wash the marked area on the forearm of the subject with known amount of fragranced product. To wash the forearm, use water having a temperature of 98° F. to 105° F. at a flow rate of 3.8 L/min. The entire forearm of the subject is wetted for 5 seconds, letting the water stream wash from the elbow to the wrist. A three piece medium cleansing puff from Zeca SRL, Italy, is wet with water for 5 seconds. Next, dispense 1.5 mL of personal care composition on inner forearm from elbow to wrist and wash inner forearm with the cleansing puff for 10 seconds over the length of the inner forearm. The lather is left on inner forearm for 15 seconds. Next, the forearm is rinsed with running water from elbow to wrist for 15 seconds. Then, the forearm is dry with a paper towel.

After the subject's forearm has been washed, a glass extraction cup, a cylinder open ended on both sides having a 2.75 cm inner diameter and a 15.9 cm² extraction area and securing straps is attached on inner forearm and is straps are secured with a strap. Next, add 5 ml of pentane (Omnisolv, high purity px0167-1) that comprises an internal standard decyl acetate at a concentration of 0.27 g/ml to the secured extraction cup. The pentane is stirred over the subject's skin surface for 30 seconds using the glass stir rod. Next, the sample obtained from the application of pentane is caught by the secured cup and then transferred to capped glass vial, such as a 20 ml scintillation vials, using a disposable glass pipette. The sample is then transferred to a standard GC-MS vial and proceeds with GC-MS analysis.

Next, the sample will be loaded and analyzed by a Gas Chromatograph (GC): Hewlett Packard (HP) model 6890 with a CIS-4 injector (Gerstel, Mulheim, Germany). The gas chromatograph column: is aJ&W DB-1 MS, 60 M×0.32 mm ID, 1.0 □m film thickness obtained from J&W Scientific of Folsom, Calif., USA. The carrier gas used is helium using a 1.5 ml/min. flow rate. The injector liner is packed with deactivated glass wool and Tenax TA having a 60/80 mesh. The detector is a model HP 5973 Mass Selective Detector obtained from Hewlett Packard, Santa Clarita, Calif., USA having a source temperature of about 230° C., and a MS Quad temperature of about 150° C. The autosampler is a Agilent 7683 series injector.

To proceed with analysis, the injector is cooled to 40° C., and a 50 □l of sample is injected to the Tenax-packed injector. A helium flow (flow rate about 30 ml/min) is used to purge the solvent. The purge time is 11 minutes. Then the injector is heated to desorb the trapped perfume compounds and GC-MS analysis run is started. A temperature program is used that comprises an initial temperature of about 50° C. which is held for 2 minutes, an increase the initial temperature at a rate of about 6° C./min until a temperature of about 285° C. is reached, and hold at about 285° C. for 10 minute.

The perfume compounds within the personal care composition are identified using the Mass Spectroscopy spectral libraries of John Wiley & Sons and the National Institute of Standards and Technology (NIST), purchased and licensed through. Hewlett Packard. The chromatographic peaks for specific ions are integrated using the Chemstation software obtained from Hewlett Packard, Santa Clarita, Calif., USA. The peak area ratio for the specific ion of the specific compound to the internal standard is calculated. The amount for each perfume component is calculated based on a calibration curve for the specific ion of the specific compound using standard amounts and the same internal standard. Deposition efficiency for the specific perfume component can then be calculated based on the amount of the personal care composition that was applied to the forearm.

### Method of Use

The personal care compositions of the present invention are preferably applied topically to the desired area of the skin or hair in an amount sufficient to provide effective delivery of the skin cleansing agent, hydrophobic material, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water. The present invention is therefore also directed to methods of cleansing the skin through the above-described application of the compositions of the present invention.

### Method of Manufacture

The multi-phase personal care compositions of the present invention may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166 issued to Thibiant, et al. on Apr. 10, 2001. The method and apparatus allows two or more compositions to be filled in a spiral configuration into a single container using at least two nozzles which fill the container, which is placed on a static mixer and spun as the composition is introduced into the container.

Alternatively, the present invention can be prepared by a method disclosed in commonly owned U.S. patent application Ser. No. 10/837,214 Publication No. 2004/0219119 A1 entitled “Visually distinctive multiple liquid phase compositions” filed by Wei, et al. on Apr. 30, 2004, published on Nov. 18, 2004. The method and apparatus allows two separate compositions to be combined in predetermined amounts, blended into a single resultant composition with visually distinct phases, and filled by one nozzle into a single container that is lowered and rotated during filling.

If the multi-phase personal care compositions are patterned, it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

### EXAMPLES

The following are non-limiting examples of the Present Invention.

TABLE 4
Examples of the Present Invention

| Ingredient Chemical INCI Name | Example 1 | Example 2 | Example 3 |
|---|---|---|---|
| Water | QS | QS | QS |
| Sodium Laureth 3 Sulphate | 4.9 | 8.2 | 9.2 |
| Cocamidopropyl Betaine | 5.7 | 1.9 | 4.0 |
| Sodium Lauroyl Sarcosinate | 0.35 | | |
| Sodium Lauroamphoacetate | — | 1.2 | 1.0 |
| Decyl Glucoside | 0.4 | — | — |
| Lauryl Glucoside | 0.5 | — | — |
| Polyquaternium-10 | 0.1 | 0.8 | |
| Citric Acid | Max 1 | Max 1 | Max 1 |
| Sodium Hydroxide | Max 0.3 | Max 0.3 | Max 0.3 |
| Sodium Sulfate | 1.2 | 2.2 | 2.0 |
| Tetrasodium EDTA | 0.15 | 0.15 | 0.15 |
| Sodium Benzoate | — | 0.25 | — |
| DMDM Hydantion | 0.3 | 0.3 | 0.3 |
| Lauryl Alcohol | 0.5 | — | — |
| Glycerin | — | 0.5 | 0.3 |
| Perfume 3 | — | 1.1 | — |
| Perfume 4 | 1.25 | — | 1.0 |
| D&C Red 33 | 0.0003 | — | — |
| FD&C Green 3 | 0.0001 | — | — |
| pH | 6.2 | 6.0 | 6.0 |

Examples 1-3 described above in Table 4 is prepared by conventional formulation and mixing techniques. First, weigh water into a suitable vessel and begin mixing with low agitation. Next, slowly disperse the polymer, Polyquatemium 10, into the water and mix for 20 minutes. Once the polymer is hydrated, slowly add sodium sulfate and begin to heat to 40° C. Once salt is dissolved, cool to 20° C. Add Sodium Laureth 3 Sulfate and citric acid. Allow to mix for approximately 20 minutes, until composition is homogeneous. Add remaining surfactants and allow to mix for approximately 30 minutes, until the composition is homogeneous. Add perfume, and dyes to the composition and mix for 30 minutes. Adjust the pH to the specified limit, mix for a further 20 minutes.

TABLE 5
Examples of the Present Invention

| | | |
|---|---|---|
| Ingredient | Example 4 | Example 5 |

I. First Phase

| | | |
|---|---|---|
| Ammonium Laureth-3 Sulfate (25% Active) | 46.7 | 52.0 |
| Citric Acid Anhydrous | 1.76 | 1.76 |
| Sodium Lauroamphoacetate (27%) | 43.47 | 35.0 |
| Trihydroxystearin (Thixcin R from Rheox) | 2.35 | 2.35 |
| Preservatives | 1.73 | 1.73 |
| Lauric Acid | 2.35 | 2.35 |
| Petrolatum | 1.64 | 1.64 |

II. Second Phase

| | | |
|---|---|---|
| Ammonium Laureth-3 Sulfate | 18 | 18 |
| Ammonium Lauryl Sulfate (25% Active) | 12 | 12 |
| Phase 1 | 42.6 | 42.6 |
| Fragrance 3 | 1.0 | |
| Fragrance 5 | | 1.2 |

Premix 1

| | | |
|---|---|---|
| Guar Hydroxypropyltrimonium Chloride | 0.3 | 0.3 |
| (N-Hance 3196 from Aqualon) | | |
| Water | QS | QS |

Premix 2

| | | |
|---|---|---|
| Petrolatum | 17.3 | 17.3 |
| Titanium Dioxide/Mica/Silica/Dimethicone | 1.5 | 1.5 |
| (SAT-Timiron Splendid Red from US Cosmetics) | | |

Examples 4-5, described above in Table 5 can be prepared by conventional formulation and mixing techniques. Phase 1 is prepared by first adding citric acid into the ammonium laureth-3 sulfate. Once the citric acid is full dissolved add the sodium lauroamphoacetate. Heat the mixture to 190 (81.8° C.)-195° F. (90.6° C.). Incorporate the trihydroxystearin fully and then add preservatives. Continue to mix as petrolatum is added. Phase 2 is prepared in a separate mixing vessel. Add ammonium laureth-3 sulfate then ammonium lauryl sulfate to mixing vessel in a water bath. To this vessel add Phase 1 with continuous mixing. Premix the guar hydroxypropyl trimonium chloride and water (Premix 1). Add Premix 1 to mixing vessel. Premix 2 is prepared by adding petrolatum into a separate mixing vessel. Heat the vessel to 190° F. Add SAT-Timiron Splendid Red and allow to mix well. Then add Premix 2 to Phase 2. Then add perfume. Keep agitation until a homogenous solution forms.

The following examples described in Table 6 are non-limiting examples of lathering cleansing phase and non-lathering structured aqueous phase compositions of the present invention.

TABLE 6
Examples of the Multi-phase Personal Care
Composition of the Present Invention

| | | | | |
|---|---|---|---|---|
| | Example 6 | Example 7 | Example 8 | Example 9 |
| Ingredient | wt % | wt % | wt % | wt % |

I. First Phase (Lathering Cleansing Phase Composition)

| | | | | |
|---|---|---|---|---|
| Miracare SLB-365 | 47.4 | 47.4 | 47.4 | 47.4 |
| (from Rhodia) (Sodium | | | | |
| Trideceth Sulfate, | | | | |
| Sodium | | | | |
| Lauramphoacetate, | | | | |
| Cocamide MEA) | | | | |
| Cocamide MEA | 3.0 | 3.0 | 3.0 | 3.0 |
| Guar | 0.7 | 0.7 | 0.7 | 0.7 |
| Hydroxypropyltrimonium | | | | |
| Chloride | | | | |
| (N-Hance 3196 from | | | | |
| Aqualon) | | | | |
| PEG 90M (Polyox WSR | 0.2 | 0.2 | 0.2 | 0.2 |
| 301 from Dow Chemical) | | | | |
| Glycerin | 0.8 | 0.8 | 0.8 | 0.8 |
| Sodium Chloride | 3.5 | 3.5 | 3.5 | 3.5 |
| Disodium EDTA | 0.05 | 0.05 | 0.05 | 0.05 |
| Glydant | 0.67 | 0.67 | 0.67 | 0.67 |
| Citric Acid | 0.4 | 0.4 | 0.4 | 0.4 |
| Perfume 2 | 1.5 | — | — | — |
| Perfume 3 | — | — | 1.1 | — |
| Perfume 4 | — | 2.0 | — | 1.3 |
| Red 7 Ca Lake | 0.01 | 0.01 | 0.01 | 0.01 |
| (From LCW) | | | | |
| Water | Q.S. | Q.S. | Q.S. | Q.S. |
| (pH) | (6.0) | (6.0) | (6.0) | (6.0) |

II. Second Phase (Non-Lathering Structured Phase Composition)

| | | | | |
|---|---|---|---|---|
| Acrylates/Vinyl | 1.0 | — | 1.0 | — |
| Isodecanoate | | | | |
| Crosspolymer | | | | |
| (Stabylen 30 from 3V) | | | | |
| Xanthan gum | 1.0 | — | 1.0 | — |
| (Keltrol CGT | | | | |
| from CP Kelco) | | | | |
| Petrolatum (Superwhite | 10 | 75 | 10 | 75 |
| Protopet from | | | | |
| Witco) | | | | |
| Mineral Oil (Hydrobrite | — | 25 | — | 25 |
| 1000PO from | | | | |
| Crompton Corp.) | | | | |
| Triethanolamine | 1.5 | — | 1.5 | — |
| Sodium Chloride | 3.5 | — | 3.5 | — |
| Perfume 3 | 1.0 | | — | — |
| Perfume 4 | — | 1.5 | — | 1.2 |
| Perfume 5 | — | — | 2.0 | — |
| Glydant | 0.37 | — | 0.37 | — |
| Water and Minors | Q.S. | — | Q.S. | — |
| (pH) | (6.0) | N/A | (6.0) | N/A |

Examples 6-9 described above can be prepared by conventional formulation and mixing techniques. The lathering cleansing phase composition can be prepared by forming the following premixes: adding citric acid into water at 1:1 ratio to form a citric acid premix, add polyox WSR-301 into glycerin at 1:3 ratio to form a polyox-glycerin premix, and add cosmetic pigment into glycerin at 1:20 ratio to form a pigment-glycerin premix and mix well using a high shear mixer. Then, add the following ingredient in the main mixing vessel in the following sequence: water, N-Hance 3196, polyox premix, citric acid premix, disodium EDTA, and Miracare SLB-365. Mix for 30 minutes, then begin heating the batch to 120° F. (48.9° C.) Add CMEA and mix until homogeneous. Then, cool the batch to ambient temperature and add the following ingredients: sodium chloride, Glydant, cosmetic pigment premix and perfume. Mix the batch for 60 minutes. Check pH and adjust pH using citric acid or caustic solution if needed.

The non-lathering structured phase (Ex 1) can be prepared by slowly adding Stabylene into water with continuous mixing. Then, add Keltrol CG-T. Heat the batch to 85° C. with continuous agitation. Then, add Superwhite Protopet. Cool down the batch to ambient temperature. Then, add Triethanolamine. The batch becomes viscous. Add sodium chloride, glydant and mix until homogeneous.

The non-lathering structured phase (Ex 2) can be prepared by adding petrolatum into a mixing vessel. Heat the vessel to 88° C. Then add mineral oil with agitation. Once homogenous, allow the vessel to cool down with slow agitation.

The lathering cleansing and non-lathering structured aqueous phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length.

TABLE 7
Examples of the Multi-phase Personal Care
Composition of the Present Invention

| | | | |
|---|---|---|---|
| | Example | Example | Example |
| | 10 | 11 | 12 |
| Ingredient | wt % | wt % | Wt % |

I. Lathering Cleansing Phase Composition

| | | | |
|---|---|---|---|
| Sodium Lauryl Sulfate | — | — | 7.7 |
| Sodium Trideceth Sulfate (Cedapol | 15.4 | 15.4 | 7.7 |
| TD-407 from Stepan) | | | |
| Sodium Lauroamphoacetate | 4.6 | 4.6 | 4.6 |
| (Miranol L-32 Ultra from Rhodia) | | | |
| Guar Hydroxypropyltrimonium | 0.7 | 0.7 | 0.6 |
| Chloride (N-Hance 3196 | | | |
| from Aqualon) | | | |
| Xanthan Gum (Keltrol 1000 from | 0.6 | 0.6 | 0.5 |
| Kelco) | | | |
| Isosteareth-2 (Hetoxol IS-2 from | 2 | 1 | 2 |
| Global 7) | | | |
| Laureth-2 (Arlypon F from Cognis) | — | 2 | — |
| Sodium Chloride | 4.25 | 4.25 | 4.25 |
| Sodium Benzoate | 0.2 | 0.2 | 0.2 |
| Disodium EDTA | 0.13 | 0.13 | 0.13 |
| Glydant | 0.37 | 0.37 | 0.37 |
| Citric Acid | 0.8 | 0.8 | 0.8 |
| Perfume 5 | 0.8 | — | — |
| Perfume 5 | — | 0.9 | |
| Perfume 3 | — | — | 1.0 |
| Water | Q.S. | Q.S. | Q.S. |
| (pH) | (5.7) | (5.7) | (5.7) |

II. Non-Lathering Structured Aqueous Phase Composition

| | | | |
|---|---|---|---|
| Acrylates/Vinyl Isodecanoate | 1.0 | 1.0 | 2.0 |
| Crosspolymer | | | |
| (Stayblen 30 from 3V) | | | |
| Xanthan Gum (Keltrol 1000 from | 1 | 1 | 1 |
| Kelco) | | | |
| Triethanolamine | 0.8 | 0.8 | 1.6 |
| Sodium Chloride | 3 | 3 | 4 |
| Glydant | 0.37 | 0.37 | 0.37 |
| Pigment | 0.01 | 0.01 | 0.01 |
| Water and Minors | Q.S. | Q.S. | Q.S. |
| (pH) | (5.7) | (5.7) | (5.7) |

Examples 10-12 described above in Table 4 can be prepared by conventional formulation and mixing techniques. Add the following ingredients into the main mixing vessel in the following sequence: water, Sodium lauryl sulfate, sodium trideceth sulfate, sodium lauroamphoacetate sodium chloride, sodium benzoate, Disodium EDTA, Glydant and salt. Start agitation of the main mixing vessel. In a separate mixing vessel, disperse N-Hance 3196 in water at 1:10 ratio and form a polymer premix. Add the completely dispersed polymer premix into the main mixing vessel with continuous agitation. Disperse the xanthan gum in the laureth-2 and isosteareth-2 and then add to the batch. Adjust the pH with the citric acid, and then add the rest of the water and perfume into the batch. Keep agitation until a homogenous solution forms.

The non-lathering structured aqueous phase can be prepared by slowly adding Stabylne 30 and xanthan gum into water in a mixing vessel. Then, add salt and neutralize with TEA. Finally, add glydant and cosmetic pigment with agitation. Mix until homogeneous.

The lathering cleansing and non-lathering structured aqueous phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length. The products remain stable at ambient for at least 180 days.

TABLE 8
Examples of In-Shower Body Lotion Compositions of the
Present Invention

| | | |
|---|---|---|
| | Ex. 13 | Ex. 14 |
| Ingredient | wt % | Wt % |

| | | |
|---|---|---|
| I. Aqueous Phase Composition | | |
| Hydroxypropyl Starch Phosphate (Structure | 3.5 | 4.0 |
| XL from National Starch) | | |
| Emulsifying Wax NF (Polawax from Croda) | 2.75 | 3.0 |
| Perfume 3 | 1.0 | — |
| Perfume 4 | — | 1.25 |
| Preservatives | 0.8 | 0.8 |
| Water | Q.S. | Q.S. |
| II. Lipid/HMIP phase Composition | | |
| Petrolatum (Superwhite Protopet from | 15 | — |
| WITCO) | | |
| Mineral Oil (Hydrobrite 1000 PO White MO from | — | 20 |
| WITCO) | | |

The personal care composition of Example 13-14, described above can be prepared by conventional formulation and mixing techniques. Prepare the aqueous phase composition by first dispersing the hydroxypropyl starch phosphate in water. Add and heat the emulsifying wax is to 160° F. (71.1° C.). Next, Place the mixing vessel in a water bath to cool to under 100° F. (37.8° C.). Add fragrance. Prepare the lipid phase by first premixing the HMIP(s) if necessary. Then, add the HMIP(s) to the lipid to the premix at 160° F. (71.1° C.). Then, add the lipid/HMIP phase to the aqueous phase (<80° F. (26.7° C.)) with increased agitation. Add preservatives and agitate until product is smooth.

The compositions illustrated in the following Examples are prepared by conventional formulation and mixing methods, an example of which is described above. All exemplified amounts are listed as weight percents and exclude minor materials such as diluents, preservatives, color solutions, imagery or conceptual ingredients, botanicals, and so forth, unless otherwise specified.

TABLE 9
Examples of the Present Invention

| Ingredient | Ex. 15 | Ex. 16 |
|---|---|---|
| Ammonium Laureth-3 Sulfate | 12 | 10 |
| Ammonium Lauryl Sulfate | 2 | 6 |
| Cocamidopropyl Betaine | 2 | — |
| Coconutmonoethanol amide (CMEA, Mona | 2 | 0.8 |
| Industries) | | |
| Cetyl alcohol | — | 0.6 |
| Ethylene Glycol Distearate (EGDS) | — | 1.5 |
| Structure Plus (National Starch) | — | 3 |
| Carbopol Aqua SF-1 (30%) (Noveon) | 3 | — |
| Polyquaterium 10, (UCARE polymer JR- | 0.25 | — |
| 30M from Amerchol) | | |
| Polymethacrylamidopropyltrimonium | — | 0.13 |
| Chloride (Polycare 133 from Rhodia) | | |
| Dow Corning 1870 (silicone nanoemulsion) | 2 | — |
| Puresyn 6 (1-decene homopolymer) | | 0.3 |
| Kathon CG (Rhom & Haas) | 0.0005 | 0.0005 |
| Disodium EDTA (Dissolvine NA-2S, Akzo Nobel) | 0.1274 | 0.1274 |
| Sodium chloride (Morton) | 0.5 | 0.7 |
| Sodium Citrate Dihydrate | 0.4 | 0.4 |
| Citric Acid (Hoffman-Laroche) | 0.15 | 0.15 |
| Perfume 2 | 2.0 | — |
| Perfume 5 | — | 1.5 |
| Water | q.s. | q.s. |

The personal care composition of Example 15-16, described above can be prepared by conventional formulation and mixing techniques. In an appropriate vessel, add distilled water and stir at an appropriate speed (100-200 ppm) using an appropriate sized stir blade. If needed, add the anionic polymer (Carbopol Aqua SF-1), cationic polymers (Polyquatemium-10, Polycare 133) and stir briefly and slowly to wet and disperse the polymer. While continuing to stir, if needed, add the citric acid solution (50%) drop wise to the mix vessel to reduce pH until solution becomes clear. Add surfactants (ALS, AE3S, CAPB,) to the mixture. Heat the mixture to 60° C. and while stirring add CMEA, EGDS, and cetyl alcohol to the mixture. Mix until homogeneous. Cool the solution to room temperature while stirring and add Silicone(s), Puresyn, Kathon, EDTA, Mackstat DM-C, D&C pigment, and perfume. Finally, adjust pH of the product within the preferred specified range of from about 5.5 to about 6.5.

TABLE 10
Examples of the Present Invention

| Ingredient | Example 17 | Example 18 |
|---|---|---|
| Stearamidopropyldimethylamine (1) | 2.0 | 1.2 |
| L-Glutamic acid (2) | 0.64 | 0.38 |
| Quaternium-18 (21) | — | 0.5 |
| Cetyl alcohol (3) | 2.5 | 2.00 |
| Stearyl alcohol (4) | 4.5 | 3.60 |
| Dimethicone blend (5) | — | 1.5 |
| Dimethicone/Cyclomethicone blend (6) | 4.2 | — |
| Benzyl alcohol (7) | 0.4 | 0.4 |
| EDTA (8) | 0.1 | 0.1 |
| Disodium EDTA (19) | — | — |
| Kathon CG (9) | 0.03 | 0.03 |
| Panthenyl Ethyl Ether (10) | 0.05 | 0.06 |
| Panthenol (11) | 0.09 | 0.05 |
| Perfume | 0.25 | 0.30 |
| Deionized Water | qs | Qs |

The personal care composition of Example 17-18, described above can be prepared by conventional formulation and mixing techniques. Examples 17-18, mix water, stearamidopropyldimethylamine, and L-glutamic acid at a temperature above 70° C. Then, add cetyl alcohol, stearyl alcohol, and benzyl alcohol with agitation. Cool down below 60° C., then add silicones, kathon, EDTA, panthenyl ethyl ether, panthenol and perfume with agitation. Then, cool down to about 30° C.

TABLE 11
Examples Bar Soaps of the Present Invention

| | Example 19 | Example 20 | Example 21 |
|---|---|---|---|
| Ingredient | % w/w | % w/w | % w/w |
| Soap | 77.95 | 63.26 | — |
| Free Fatty Acid | 5.70 | 4.29 | — |
| Potassium Alkyl Sulfate | — | 10.0 | — |
| Sodium Laureth 3 Sulfate | — | 3.0 | — |
| Sodium Cocoyl Isethionate | — | — | 22.0 |
| Paraffin | — | — | 19.4 |
| Sodium Alkyl Glyceryl Ether | — | — | 17.2 |
| Sulfonate | | | |
| Sodium Isethioate | — | — | 3.0 |
| PEG 90M | — | — | 0.05 |
| Sodium Soap | — | — | 4.0 |
| Magnesium Soap | — | — | 6.5 |
| Fatty Acid | — | — | 9.0 |
| Micellaneous | — | — | 9.0 |
| Water | 11.50 | 7.5 | 5.0 |
| Sodium Chloride | 1.10 | 0.9 | |
| Titanium Dioxide | 0.25 | 0.25 | 0.25 |
| Perfume 3 | 1.0 | — | 1.1 |
| Perfume 4 | — | 0.9 | — |

The personal care composition of Example 19-21, described above can be prepared by conventional formulation and mixing techniques for bar soaps. Mix base fragrance into dried soap noodles in an amalgamator. The material is processed, for example by milling through a three roll soap mill, to obtain a homogeneous mixture of perfume and soap flakes. Then the material is processed on a plodder and is stamped into a soap bar.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising 3% or less, by weight of said personal care composition, of a perfume; and at least 0.27%, by weight of the personal care composition, of perfume raw materials having a Kovat's Index of greater than 1700.

2. The personal care composition of claim 1, wherein 0.5% of said composition comprises perfume raw materials have a ClogP of greater than about 3.5.

3. The personal care composition of claim 1, wherein said personal care composition comprises 2.5% or less, by weight of said personal care composition, of a perfume.

4. The personal care composition of claim 1, wherein said personal care composition comprises 1.5% or less, by weight of said personal care composition, of a perfume.

5. The personal care composition of claim 1, wherein said personal care composition comprises at least 0.4%, by weight of said personal care composition, of said perfume raw materials having a Kovat's Index of greater than 1700.

6. The personal care composition of claim 1, wherein said personal care composition comprises at least 0.7%, by weight of said personal care composition, of said perfume raw materials having a Kovat's Index of greater than 1700.

7. The personal care composition of claim 1, wherein said cleansing phase provides a Yield Stress of greater than about 1.5 Pascal.

8. The personal care composition of claim 1, wherein said personal care composition is selected from the group consisting of bar soap, body wash, shampoo, conditioner, in-shower body moisturizer and mixtures thereof.

9. A multi-phase, personal care composition comprising: a first phase and a second phase; said multi-phase personal care composition comprising a 3% or less, by weight of said personal care composition, of a perfume; and at least 0.27%, by weight of the personal care composition, of perfume raw materials having a Kovat's Index of greater than 1700.

10. The multi-phase personal care composition of claim 1, wherein said personal care composition comprises 2.5% or less, by weight of said personal care composition, of a perfume.

11. The multi-phase personal care composition of claim 1, wherein said personal care composition comprises 1.5% or less, by weight of said personal care composition, of a perfume.

12. The multi-phase personal care composition of claim 9, wherein said personal care composition comprises at least 0.4%, by weight of said personal care composition, of said perfume raw materials having a Kovat's Index of greater than 1700.

13. The multi-phase personal care composition of claim 9, wherein said personal care composition comprises at least 0.7% by weight of said personal care composition, of said perfume raw materials having a Kovat's Index of greater than 1700.

14. The multi-phase, personal care composition of claim 9, wherein said first phase comprises said perfume raw materials.

15. The multi-phase, personal care composition of claim 9, wherein said cleansing phase provides a Yield Stress of greater than about 1.5 Pascal.

16. The multi-phase personal care composition of claim 9, wherein said personal care composition is selected from the group consisting of bar soap, body wash, shampoo, conditioner, in-shower body moisturizer and mixtures thereof.

17. The multi-phase, personal care composition of claim 9, wherein said second phase is selected from the group consisting of a cleansing phase, a benefit phase, a non-lathering structured aqueous phase, and combinations thereof.

18. The multi-phase, personal care composition of claim 9, wherein said second phase is a benefit phase comprises hydrophobic material with a Vaughan Solubility Parameter of from about 5 to about 15.

19. The multi-phase, personal care composition of claim 9, wherein said first phase is visually distinct from said second visually distinct phase.

20. The multi-phase, personal care composition of claim 9, wherein said first phase is a cleansing phase comprising surfactant and water.
